(19) United States
(12) Patent Application Publication
(10) Pub. No.: US 2024/0382334 A1
(43) Pub. Date: Nov. 21, 2024

(54) Off-loading Wrist Brace

(71) Applicant: AR Orthopedics, LLC, Newberry, FL (US)

(72) Inventors: David Alvarez, Tallahassee, FL (US); Sharon Rosenberg, Newberry, FL (US)

(21) Appl. No.: 18/688,969

(22) PCT Filed: Sep. 6, 2022
(86) PCT No.: PCT/US2022/042669

§ 371 Date: Mar. 4, 2024

Related U.S. Application Data
(60) Provisional application No. 63/260,917, filed on Sep. 3, 2021.

(51) Int. Cl. A61F 5/01

(52) U.S. Cl. CPC A61F 5/013

(57) ABSTRACT

An off-loading wrist brace featuring a linear configuration, a split linear configuration, or a glove configuration with a thumb hole, pisiform hole, purlicue saddle, and closure components. The brace compresses to reduce repetitive movement of the carpal ligament while reducing compression of the pisiform to reduce injury and pain. The brace is removably affixed by placing the thumb through the thumb hole, wrapping the brace body around the wrist to attach the closure component at the tapered distal end to the closure component at the rounded proximal end.

### CROSS REFERENCE TO RELATED APPLICATION

This application is a national stage application of PCT application number PCT/US22/42669 filed on Sep. 6, 2022, claiming priority to U.S. provisional application No. 63/260,917 entitled Off-loading Wrist Brace and having a filing date of Sep. 3, 2021, the entire contents of which are incorporated by reference.

### FIELD OF THE DISCLOSURE

The present disclosure relates to a wrist wrap or brace. The brace supports the wrist, off-loads pressure from the pisiform, and reduces ulnar nerve palsy and pisiform fracture and pisiform-ligament complex syndrome, reduces overuse-related pain in the hand and wrist, and reduces arthritis. The wrist wrap or brace can be worn during activity.

### BACKGROUND OF THE DISCLOSURE

Pisiform fractures account for up to 3% of carpal injures. The pisiform is a sesamoid bone that resembles a pea and is located in the proximal ulnar carpal row. The pisiform is important because it serves as the attachment for the flexor carpi ulnaris tendon and is the origin site of the abductor digiti minimi muscle. In addition, the pisiform forms the ulnar wall of Guyon's tunnel, an area of compression to the ulnar nerve beneath the hamate, the bone above the pisiform.

The pisiform is prone to injury from direct trauma to the volar ulnar aspect of the wrist. Strong hyperextension of the wrist and the pull of the flexor carpi ulnaris can cause a chip avulsion. These fractures present with acute tenderness over the pisiform, and a person may develop associated ulnar nerve palsy.

Wrist braces are commonly used by individuals having various wrist injuries or pain and may be worn for weeks or months as a type of conservative treatment. Carpal tunnel syndrome or median nerve compression is commonly treated first by wearing a wrist brace to stabilize the wrist and to reduce repetitive movements that cause median nerve compression which consequently reduces pain, tingling, and numbness.

The often rigid and compressive nature of traditional wrist braces, orthosleeves, gloves, and wraps helps with stabilization and reducing compression on the median nerve but also induces compression on the pisiform and hamate, potentially causing a fracture. For example, a traditional brace has a thick, rigid shield that extends from the palm of the hand up to the mid forearm and is secured around the arm with hook-and-loop closures, compressing the hand, wrist, and arm. Other traditionally used wrist wraps compress the wrist bones bringing the thumb closer to the pisiform.

A traditional alternative to wrist braces is resting the wrist on padding such as a gel foam, but this is not as effective for reducing the underlying cause of median nerve compression, repeated movements during deskwork, and any static activity where the ulnar side of the wrist. When conservative approaches are ineffective, surgery may be required.

### SUMMARY OF THE DISCLOSURE

What is needed is a wrist brace that stabilized the wrist with compression to reduce instances of repeated movements that injure the carpal ligament and median nerve while off-loading pressure from the pisiform which reduces compression of the flexor carpi ulnaris and ulnar nerve and reduces the risk of injury to the pisiform. In other words, pisiform ligament complex syndrome and piso-triquetal arthrosis is substantially avoided. Pain and arthritis are reduced while permitting wrist and hand movement during activities such as cleaning, sports, writing, and typing.

In one embodiment, the brace has a linear body configuration comprising a rounded proximal end and a tapered distal end and a top side and a bottom side. The rounded proximal end comprises a thumb opening, a purlicue saddle, and a first closure component. The mid portion comprises an offload target. The tapered distal end comprises a second closure component.

In another embodiment, the brace body comprises a single strap having a first closure component and a corresponding second closure component. In an alternative embodiment, the brace body comprises two straps, each strap having a first closure component and a corresponding second closing component.

In one embodiment, the brace is constructed from a neoprene, foam, latex, silicone, cotton, wool, polyester, elastic, acetate, acrylic, and combinations thereof. In one embodiment, the brace top side has a breathable material such as mesh, and the bottom side has a stretch material such as neoprene. In a further embodiment, the bottom side of the brace has an interior layer comprising a receiving texture and an exterior layer comprising neoprene.

In another embodiment, the brace mid portion comprises a receiving texture on the bottom side interior that accommodates placement of an offload target having a corresponding grip texture.

In one embodiment, the thumb opening of the brace has an annular configuration. In another embodiment, the thumb opening of the brace has contoured configuration integrated with the purlicue saddle. In another embodiment, the thumb opening of the brace has a sheath configuration.

In one embodiment, the purlicue saddle of the brace comprises padding. In another embodiment, the purlicue saddle of the brace comprises trim. In an alternative embodiment, the purlicue saddle of the brace comprises a wedge.

In one embodiment, the brace offload target comprises a border, a first side, a second side, an opening edge, and an opening. In one embodiment of a removably attached offload target, the first side has a smooth texture, and the second side has a grip texture. In one embodiment of either a removably attached or fixedly attached offload target, the opening has an exposed configuration or a concealed configuration.

In one embodiment, the offload target is fixedly attached to the proximal end. In an alternative embodiment, the offload target is removably attached and is positioned on and removed from the mid portion bottom interior.

Pisiform ligament complex syndrome and carpal ligament and median nerve injury are avoided while stabilizing the wrist with a brace having a linear body comprising a rounded proximal end and a tapered distal end and a top side and a bottom side and where the rounded proximal end comprises a thumb opening, a purlicue saddle, a palm saddle, and a first closure component, and where the mid portion comprises an offload target, and where the tapered distal end comprises a second closure component.

### BRIEF DESCRIPTION OF THE DRAWINGS

The accompanying drawings that are incorporated in and constitute a part of this specification illustrate several embodiments of the disclosure. Together with the description, the drawings explain the principles of the disclosure.

FIG. 1 illustrates a simplified anatomy of the wrist and hand.

FIG. 2 illustrates an exemplary brace in a linear body configuration.

FIG. 3 illustrates a top view of an exemplary brace having a linear body configuration being worn.

FIG. 4 illustrates a bottom view of an exemplary brace being worn.

FIG. 5 illustrates a full front view of an exemplary brace having a linear split body configuration.

FIG. 6 Illustrates a bottom view of an exemplary brace having a linear split body.

FIG. 7 illustrates a bottom exterior perspective view of a distal end of a fastened brace.

FIG. 8 illustrates a perspective top side exterior view of a fastened brace.

FIG. 9 illustrates a top view of an exemplary brace having a glove body configuration.

FIG. 10 illustrates a bottom view of an exemplary brace having a glove body.

FIG. 11 illustrates an interior top and bottom view of an exemplary brace having a glove body.

FIG. 12 illustrates a top view and a bottom view of an exemplary offload target.

FIG. 13 illustrates an interior top and bottom view of an exemplary brace having a glove body with offload targets.

### NUMERALS OF THE FIGURES

1. brace
3. Proximal end
5. Distal end
7. First closure component
9. Second closure component
11. Thumb opening
13. Purlicue saddle
15. Mid section
17. body
19. Offload target
21. border
23. edge
25. opening
27. thumb
29. wrist
33. palm
35. top side
37. trim
41. receiving texture
43. bottom side
45. grip texture
46. inner layer
47. Exterior
49. interior
51. Closure strap
52. Finger opening
53. Top exterior
55. Bottom exterior
57. Bottom interior
59. Top interior
61. Receiving texture
63. First side
65. Second side

### DETAILED DESCRIPTION

The present disclosure provides generally for an off-loading wrist brace. Hand and wrist ligament strain and injury are common and result from repeated movements from deskwork, typing, writing, or drawing. One of the most popular ailments is carpal tunnel syndrome where the medial nerve and the carpal ligament are indicated. Stabilizing the wrist and reducing the repetitive movements by wearing a wrist brace is a common non-invasive therapy. However, whole compression of the wrist and the related bones and ligaments is detrimental to nearby bones, ligaments, and nerves.

The pisiform bone is a small bone in the wrist and forms the border of the carpal tunnel. This is problematic because treatment of carpal tunnel syndrome with a brace, effectively requires compression. The pisiform does not need to be compressed and should not be compressed. The additional and unnecessary pressure on the ulnar nerve and flexor carpi ulnaris can result in increased pain, tenderness, palsy, fracture, and sometime permanent impairment that requires surgery.

The off-loading brace of the disclosure provides for simultaneous stabilization of the wrist for treatment of ailments such as carpal tunnel syndrome while also off-loading pressure from the pisiform by including a pisiform hole. In some preferred embodiments, the brace contains contours spanning from an offloading target border to an opening edge to disperse pressure and provide additional comfort. The off-loading brace has linear, split linear, and glove configurations that reduce pain and support the wrist. The various brace configurations also have offload targets that are modular, providing more customized and additional support and offloading. In addition, the braces of the disclosure relieve pressure on scar tissue from previous surgeries and prevent agitation of other nerves and tendons of the hand and wrist.

In the following sections, detailed descriptions of examples and methods of the disclosure will be given. The description of both preferred and alternative examples are exemplary only, and it is understood that to those skilled in the art that variations, modifications, and alterations may be apparent. It is therefore to be understood that the examples do not limit the broadness of the aspects of the underlying disclosure as defined by the claims.

### Detailed Descriptions of the Drawings

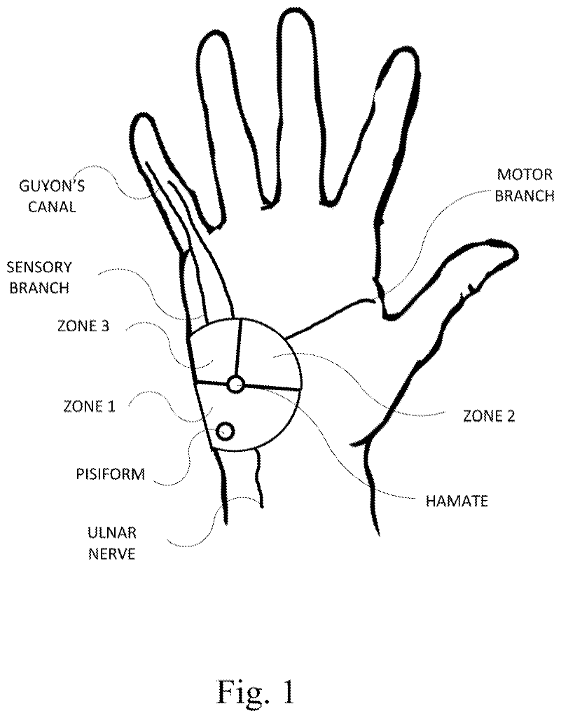

Referring now to FIG. 1, a simplified anatomy of the wrist and hand is shown. The major relevant parts shown are the Guyon's canal, the hamate, the pisiform, and the relevant nerves and nerve areas divided into zones 1-3. Zone 1 contains the ulnar nerve. Zone 2 contains the motor branch. Zone 3 contains the sensory branch. Traditional wrist braces compress the ulnar nerve but also unnecessarily compress the pisiform and/or the hamate which can lead to pain, palsy, fracture, or other damage requiring surgery or further therapy.

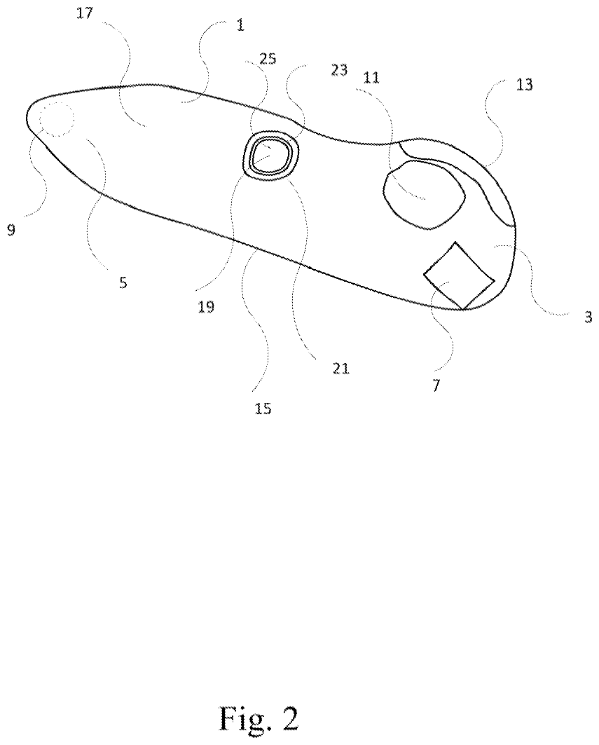

Referring now to FIG. 2, an exemplary brace in a linear configuration is shown that results in reduced pain while supporting the wrist. The brace is comprised of a substantially linear body having a top side and a bottom side (underneath; not shown) and a tapered distal end and a rounded proximal end. The rounded proximal end has a purlicue saddle and a thumb opening such as a thumb hole, a thumb contour, or a thumb sheath. In some embodiments, the purlicue saddle may be padded with a saddle material comprising cotton, wool, latex, foam, silicone, or combinations thereof. Exemplary braces may be constructed from neoprene, latex, cotton, wool, elastic, acrylic, polyester, or any other suitable material with sufficient elasticity and structural integrity to snuggly wrap around the wrist and provide compression.

The brace may also comprise closure components such as a hook-and-loop closure, adhesive, lace-up, button, snap, hook-and-eye closure, or other attachment means. In preferred embodiments, the brace comprises a first closure component and a second closure component (on the bottom side). The first closure component may be located at or near the tapered distal end. The second closure component may be located at or near the rounded proximal end.

The brace also has a middle section between the tapered distal end and the rounded proximal end where an offload target such as a pisiform hole is located. In some embodiments, the offload target has a surrounding area or a border of graduated or contoured thickness from the border to an edge outlining an opening of the pisiform hole. However, in alternative embodiments, the offload target may be substantially flush with the surface of the brace body bottom side. In alternative embodiments, the offload target may have padding or cushioning in a non-contoured or graduated thickness. In some embodiments the graduated thickness or contour may increase the further away from the opening of the offload target. In some embodiments, the contouring or graduated thickness may be removable such as in the form of adhesive padding.

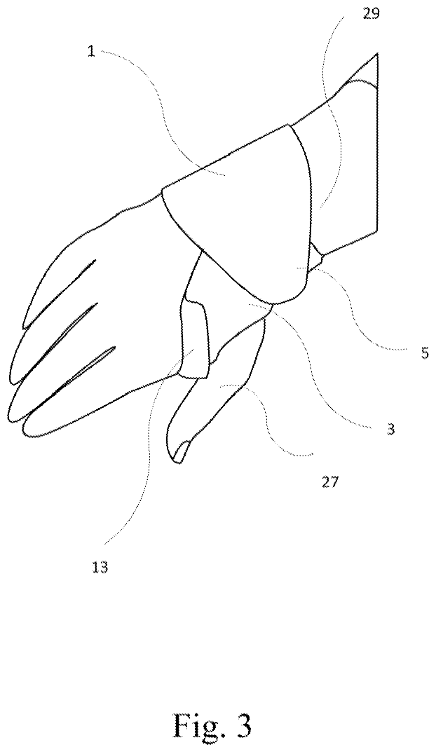

Referring now to FIG. 3, a top view of an exemplary brace being worn is shown that results in reduced pain while supporting the wrist. In particular, the top view shows the brace fitted around the wrist, providing the necessary stabilization and compression. The purlicue saddle padding extends over the edge of the purlicue saddle of the brace body in preferred embodiments. In some embodiments, the padding may be interchangeable, and in some instances, the brace may be worn without any padding.

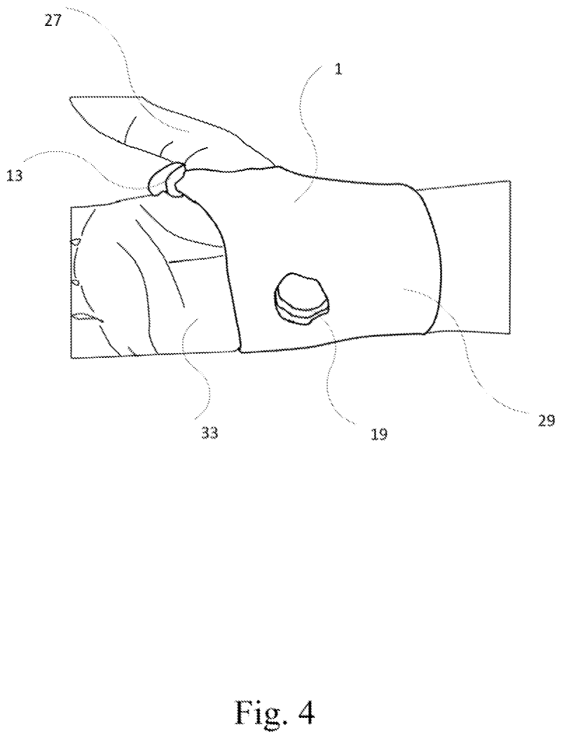

Referring now to FIG. 4, a bottom view of an exemplary brace being worn is shown that results in reduced pain while supporting the wrist. The figure demonstrates how the brace compresses the wrist to stabilize the ulnar ligament and de-compresses the pisiform to prevent pisiform-ligament complex syndrome. Once the brace is obtained, the wearer inserts the thumb through the thumb hole, placing the purlicue saddle along the purlicue. The wearer or a third person applying the brace grabs the tapered distal end, wrapping the tapered distal end around the wrist. The person may pull harder or softer depending on the desired level of compression. The first closure component of the tapered distal end is joined to the second closure component of the rounded proximal end. As a result, the wrist is covered substantially by the brace while the pisiform is exposed.

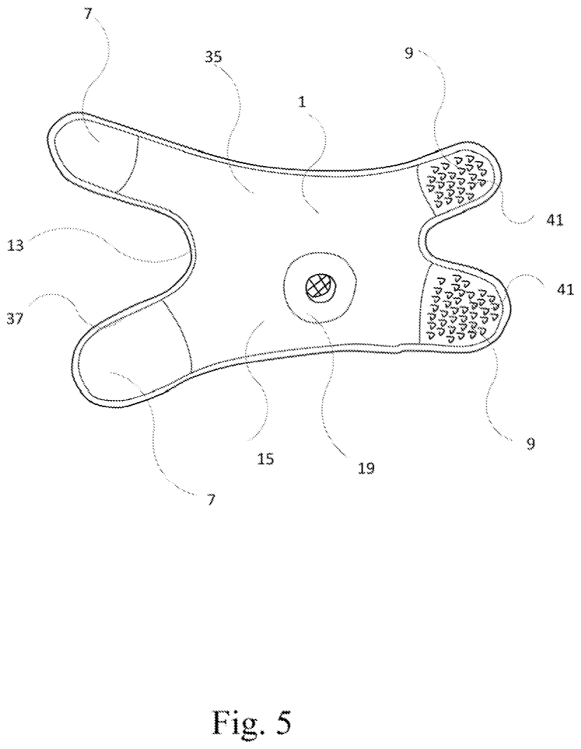

Referring now to FIG. 5, a full front view of an exemplary brace having a linear split body configuration is shown that results in reduced pain while supporting the wrist. The brace having a linear split body configuration shows a top side, trim spanning a perimeter of the body, a plurality of first closure components, a plurality of corresponding second closure components, and a mid section between the closure components. The mid section comprises an offload target. The purlicue saddle is contoured at the curvature of the trim between the plurality of first closure components. The plurality of second closure components comprises a receiving texture on the top side.

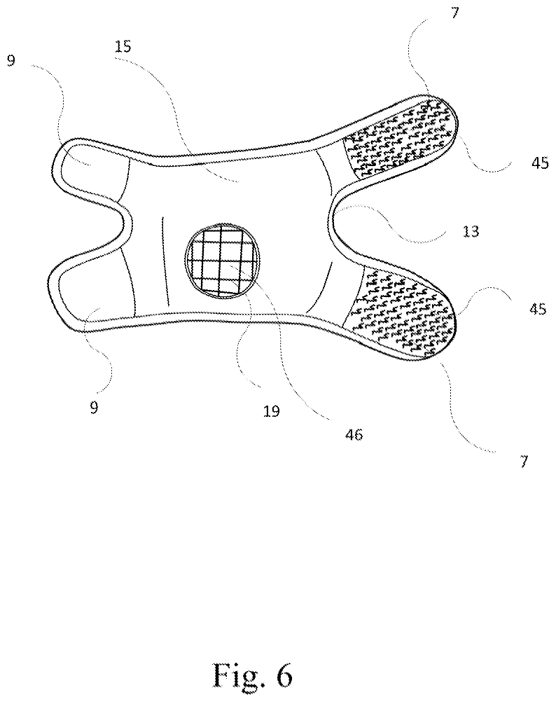

Referring now to FIG. 6, a bottom view of an exemplary brace having a linear split body is shown that results in reduced pain while supporting the wrist. This figure shows a bottom side that corresponds to the top side of the exemplary brace shown in FIG. 5. Notably, the plurality of first closure components comprises a grip texture configured to attach to the receiving texture on the top side of the plurality of second closure components. Furthermore, in one embodiment, the offload target may have an inner layer. The inner layer spans the opening of the offload target, have a concealed target as opposed to an open target that lacks an inner layer. The inner layer may be a mesh, matrix, gel, spandex, rubber, foam, or hollow space covered by fabric.

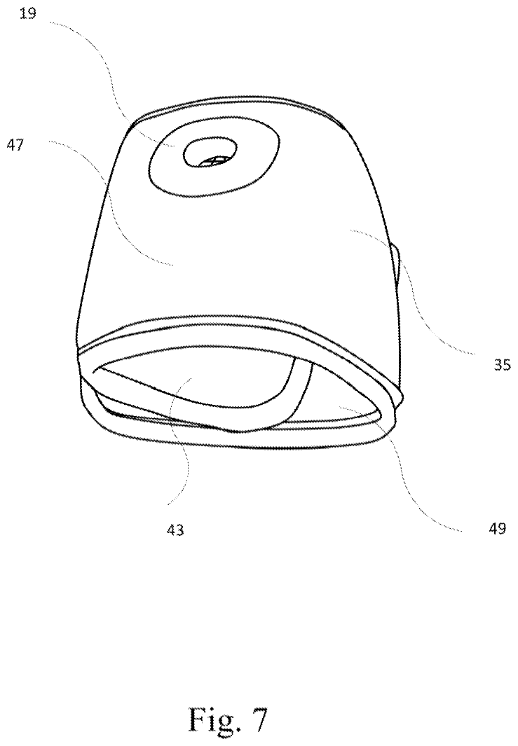

Referring now to FIG. 7, a bottom exterior perspective view of a distal end of a fastened brace is shown that results in reduced pain while supporting the wrist. The brace in FIG. 7 demonstrates how exemplary linear braces appear when the first closure components and second closure components are attached. The exterior and interior of the brace are shown along with the offload target.

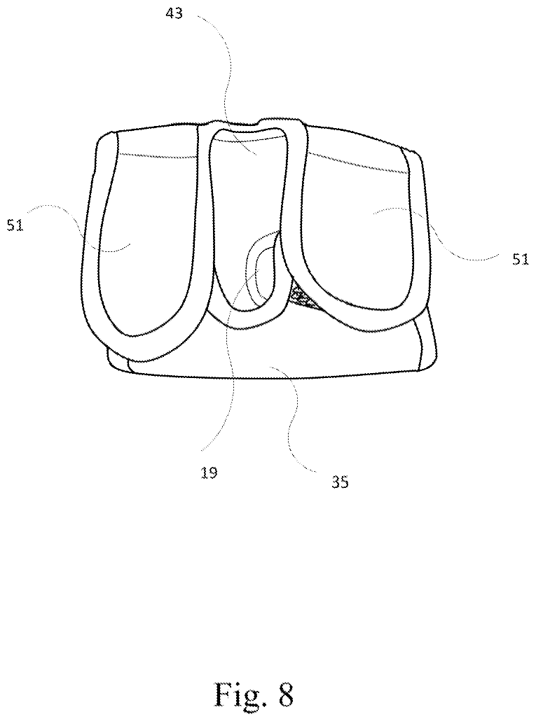

Referring now to FIG. 8, a perspective top side view of a fastened brace is shown that results in reduced pain while supporting the wrist. The brace in FIG. 8 demonstrates how exemplary linear and linear split braces appear when the first closure components and second closure components are attached. The exterior and interior are shown along with the offload target. The offload target may be flush with either the exterior or interior or both or may be raised or protruding from the interior or exterior or both, providing a customized or modular configuration that offloads pressure while stabilizing the wrist.

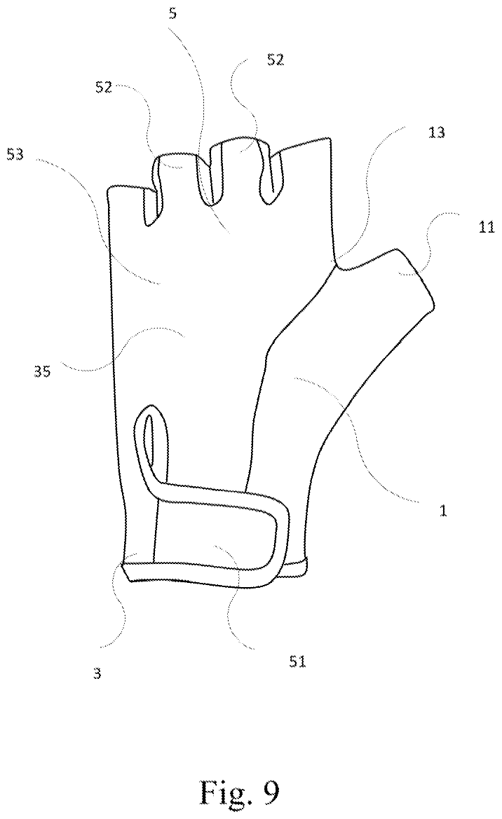

Referring now to FIG. 9, at top view of an exemplary brace having a glove body configuration is shown that results in reduced pain while supporting the wrist. The exemplary brace is shown having a proximal end where a closure strap is placed. The closure strap at the proximal end provides wrist compression. The brace distal end comprises at least one finger opening. In preferred embodiments, a glove configuration may have 4 finger openings and a thumb opening in a sheath configuration. The purlicue saddle may have a wedge configuration placed between the thumb opening and the finger opening. The exemplary brace exhibits a top exterior of a top side.

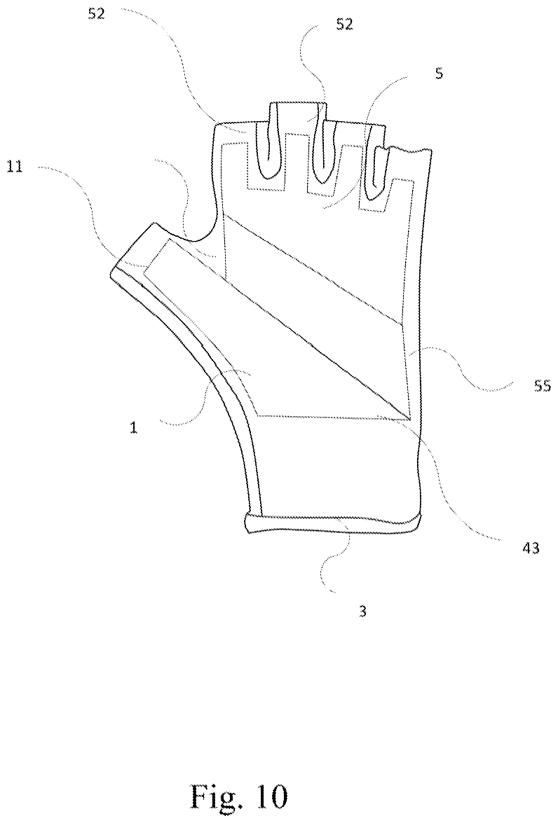

Referring now to FIG. 10, a bottom view of an exemplary brace having a glove body is shown that results in reduced pain while supporting the wrist. The exemplary brace of FIG. 10 exhibits the bottom exterior of the bottom side of the exemplary brace of FIG. 9.

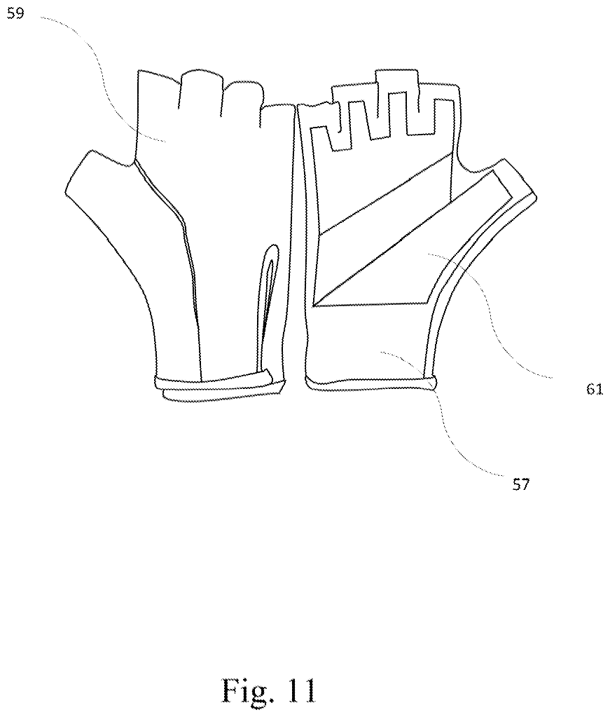

Referring now to FIG. 11, an interior top and bottom view of an exemplary offload target is shown that results in reduced pain while supporting the wrist. In one embodiment, the bottom interior and the top interior may be composed of differing materials. For example, the top interior may be composed of a breathable mesh or lightweight fabric while the bottom interior may be composed of a heavier fabric or padded multi-layer material to rest against the palm. Furthermore, one embodiment may comprise a receiving texture on all or part of the bottom interior. The receiving texture may correspond to hook and loop closure materials.

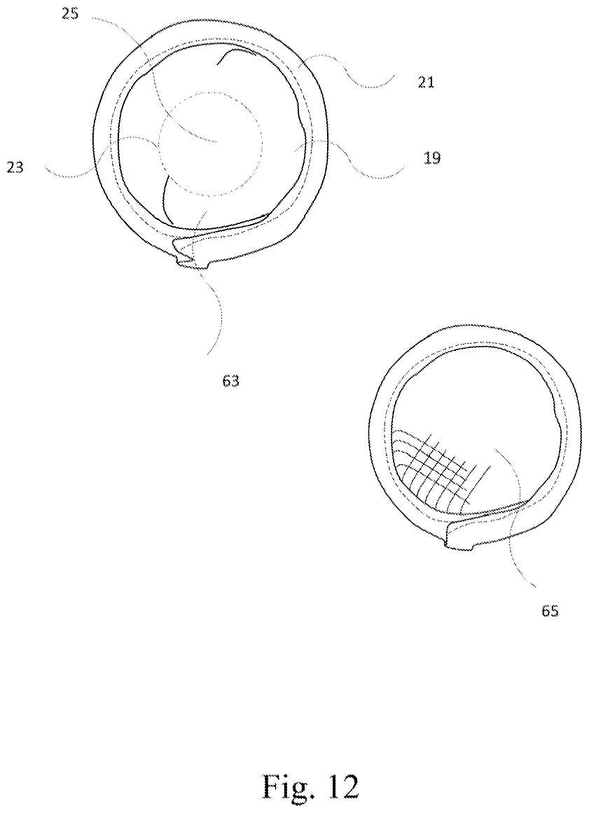

Referring now to FIG. 12, a top view and a bottom view of an exemplary offload target is shown. Exemplary offload targets may have a round or polygonal shape such as a triangle, square, or octagon that has a border spanning the circumference or perimeter. The offload target has an opening defined by an edge. The offload target may have an exposed configuration where the opening reveals or shows or exposes the wearer's skin or may have a concealed configuration where the opening comprises an inner layer as discussed in FIG. 6. Exemplary offload targets comprise a first side and a second side. A first side has a non-grip texture, and the second side has a grip texture to correspond to the receiving texture of the bottom interior of a brace.

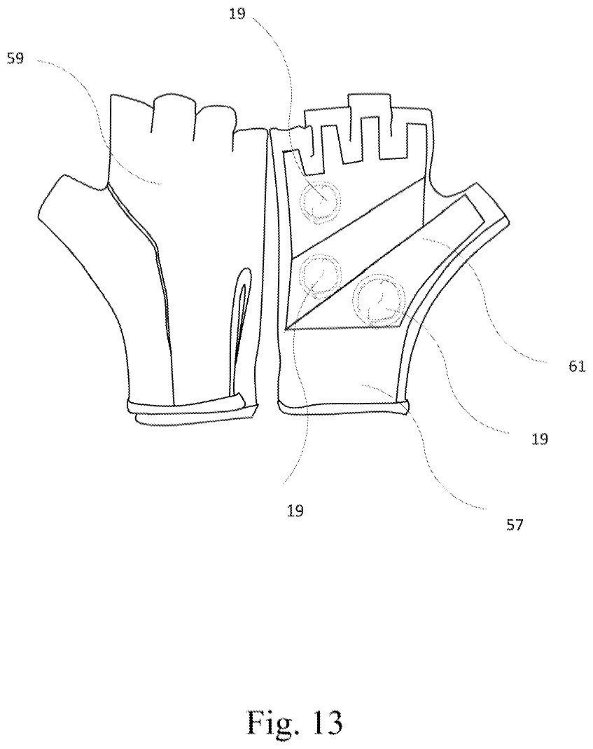

Referring now to FIG. 13, an interior top and bottom view of an exemplary brace having a glove body with offload targets is shown that results in reduced pain while supporting the wrist in a modular configuration that is customizable. In this example, a brace in a glove configuration is shown with a plurality of removably attached offload targets such as those demonstrated in

FIG. 12. However, braces in a linear or a split linear configuration may also be utilized with removably attached offload targets.

### Conclusion

A number of embodiments of the present disclosure have been described. While this specification contains many specific implementation details, these details should not be construed as limitations on the scope of any disclosures or of what may be claimed.

Certain features that are described in this specification in the context of separate embodiments can also be implemented in combination in a single embodiment. Conversely, various features that are described in the context of a single embodiment can also be implemented in combination in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order show, or sequential order, to achieve desirable results. In certain implementations, multitasking and parallel processing may be advantageous. Nevertheless, it will be understood that various modifications may be made without departing from the spirit and scope of the claimed disclosure.

What is claimed is:

1. An off-loading wrist brace, comprising
a. A body having a top side and a bottom side and a distal end and a proximal end,
b. Wherein the proximal end features a purlicue saddle, and a thumb hole, and
c. Wherein a middle section between the distal end and the proximal end comprises an offload target comprising a border, an edge, and an opening.

2. The brace of claim 1 further comprising a first closure component.

3. The brace of claim 1 further comprising a second closure component.

4. The brace of claim 2 further comprising a second closure component

5. The brace of claim 1 further comprising an interior bottom side, and interior top side, an exterior top side, and an exterior bottom side.

6. The brace of claim 4 wherein the first closure component and the second closure component provide wrist stability.

7. The brace of claim 1 wherein the body is constructed from neoprene.

8. The brace of claim 1 wherein the purlicue saddle is padded with a saddle material comprising cotton, wool, latex, foam, silicone, or combinations thereof.

9. The brace of claim 1 wherein the offload target has a surrounding area of graduated or contoured thickness.

10. The brace of claim 1 wherein the offload target is fixedly integrated into the mid section of the brace.

11. The brace of claim 5 wherein the offload target is removably attached to the interior bottom side.

12. The brace of claim 10 wherein the brace interior bottom side comprises a receiving texture.

13. The brace of claim 1 wherein the brace is configured to compress the wrist to stabilize the ulnar ligament and de-compressing the pisiform to prevent pisiform-ligament complex syndrome, the brace comprising
a. body having a top side and a bottom side and a tapered distal end and a rounded proximal end,
b. a first closure component,
c. a purlicue saddle,
d. a thumb hole, and
e. an offload target.

14. The brace of claim 13 wherein the brace body has a linear configuration.

15. The brace of claim 13 wherein the brace body has a glove configuration.
